(19) United States
(12) Patent Application Publication
(10) Pub. No.: US 2026/0283598 A1
(43) Pub. Date: Sep. 24, 2026

(54) METHODS OF PRODUCING THREE-DIMENSIONAL ARTERIAL MORPHOLOGICAL GEOMETRIES USING DUPLEX-ULTRASOUND BRIGHTNESS-MODE IMAGES

(71) Applicant: The Trustees of Indiana University, Bloomington, IN (US)

(72) Inventors: Huidan Yu, Indianapolis, IN (US); Alan P. Sawchuk, McCordsville, IN (US); David M. Rollins, Martinsville, IN (US)

(21) Appl. No.: 19/472,424

(22) PCT Filed: Apr. 4, 2024
(86) PCT No.: PCT/US2024/023010

§ 371 Date: Oct. 3, 2025

Related U.S. Application Data
(60) Provisional application No. 63/494,330, filed on Apr. 5, 2023.

(51) Int. Cl. A61B 8/00; A61B 8/06; G06T 7/00; G06T 17/00; G06V 10/77; G06V 10/774; G06V 10/82; G16H 30/40

(52) U.S. Cl. CPC A61B 8/5223; A61B 8/06; G06T 7/0014; G06T 17/00; G06V 10/7715; G06V 10/774; G06V 10/82; G16H 30/40; G06T 2200/04; G06T 2207/10136; G06T 2207/20036; G06T 2207/20081; G06T 2207/20084; G06T 2207/30101; G06T 2210/41; G06V 2201/03

(57) ABSTRACT

Methods of constructing three-dimensional arterial morphological geometries from two-dimensional images produced by duplex-ultrasound brightness-mode processes. Ultrasound cine looping is used in order to produce multiple images from multiple directions, including longitudinal and transverse directions of an artery, and three-dimensional geometries are produced from the aforementioned images by an image-based deep-learning model.

### CROSS-REFERENCE TO RELATED APPLICATIONS

This application claims the benefit of U.S. Provisional Application No. 63/494,330 filed Apr. 5, 2023, the contents of which are incorporated herein by reference.

### BACKGROUND OF THE INVENTION

The invention generally relates to medical imaging techniques. The invention particularly relates to integrated medical imaging, engineering, and morphological analysis techniques that are capable of revealing three-dimensional (3-D) morphological abnormalities of diseased vessels in support of cardiovascular diagnosis.

Cardiovascular diseases are the leading cause of morbidity, mortality, and disability in America. Approximately 850,000 deaths are caused each year by heart attacks due to coronary arterial disease (CAD), incurring an annual medical cost of $351 billion. Carotid arterial stenosis (CAS), the narrowing or constriction of carotid arteries, is the leading cause of large-vessel ischemic strokes, ischemic strokes being strokes produced by a lack of blood flow rather than bleeding. Commensurately, approximately 800,000 Americans have new or recurrent strokes each year, resulting in $34 billion in healthcare costs. Peripheral vascular diseases (PAD), a condition in which narrowed arteries reduce blood flow to the arms or legs, affect 8-12 million Americans, resulting in approximately 185,000 amputations each year and incurring healthcare costs of approximately $13.7 billion.

Inadequate blood flow is the most common cause of all of these diseases. Such inadequate blood flow, and associated vascular diseases, are typically caused by a progressive reduction (stenosis) in the artery lumen. The progression is often silent and asymptotic. Inadequate blood flow can be directly detected by indicators including insufficient blood flow rates, abnormal pressure gradients, elevated wall-shear stress (WSS), excessive kinetic turbulent energy, and other measures known to those skilled in the art. However, many of these important indicators cannot be obtained in the current standard of care (SOC), thereby negatively impacting timely diagnosis and decision-making regarding treatment methods.

Hemodynamic indicators of blood flow abnormalities can be obtained by a medical engineering technique commonly known as image-based computational hemodynamics (ICHD). ICHD is a proven capability for noninvasive and personalized quantification and virtualization of such hemodynamic abnormalities, and therefore can potentially provide accurate lesional evaluations and thereby facilitate proper treatment. FIG. 1 illustrates tasks commonly involved in ICHD, those tasks including image segmentation of the diseased artery using medical imaging data, modeling, and computation of hemodynamics in four dimensions (time and space), and post processing to retrieve hemodynamic abnormality indicators.

Studies have demonstrated that ICHD could effectively reveal how the velocity and pressure vary over time and space in diseased arteries. FIG. 2 illustrates a nonlimiting application of ICHD, wherein the pressure ratio between the flow before (proximal) and past (distal) an arterial stenosis (narrowing), commonly referred to as the fractional flow reserve (FFR), determines the hemodynamic severity of the stenosis and therefore the coronary disease. FFR currently provides the highest standard of diagnosis of myocardial ischemia. Other examples include trans-stenotic pressure gradient measurement to assess the severity of non-coronary arterial stenosis and WSS analysis to predict the rupture risk of an aneurysm. However, all of these applications, and indeed the entire ICHD methodology, are entirely dependent upon accurate, thorough medical imaging data of patients' cardiovascular and arterial systems.

Existing cardiovascular imaging methods can themselves indirectly and partially detect inadequate blood flow. Three-dimensional (3-D) medical imaging techniques include computed tomography angiography (CTA) and magnetic resonance angiography (MRA), which are capable of illustrating anatomical structure of vessels in patients and may be applied to map cardiovascular and arterial systems. FIGS. 3A and 3B are examples of 3-D anatomical carotid arteries anatomically extracted from neck CTA and MRA images. Specifically, CTA scans involve injecting an iodine-based contrast agent into a patient's arterial system, employing a rotating X-ray tube to provide multiple X-ray images from various angles of a patient's anatomy, and using the “illumination” provided by the contrast agent to effectively map the patient's arterial system. CTA scans are well known to those skilled in the art of cardiovascular imaging, diagnosis, and treatment, and therefore are not discussed in any detail here.

CTA scans provide a gold standard for illustrating 3-D cardiovascular structures in patients and revealing morphological abnormalities. Indeed, leveraging the full diagnostic capabilities of ICHD is heavily, if not entirely, reliant on CTA imaging data. However, CTA scans are not a preferred first-line diagnostic tool in practice due to its high cost, radiation risk, and the potential side effects of agent injection. CTA scans cost approximately ten times as much as comparable DUS tests. Furthermore, the effective radiation dose to a patient undergoing a single CTA scan ranges from 1-15 millisieverts (mSv), slightly less than the average radiation of 5-20 mSv received by atomic bomb survivors who historically experienced significantly higher risks of several types of cancers. As a result, CTA scans are not a preferable imaging method, particularly as a first-line diagnostic test for vascular diseases.

Rather, the prevailing first-line diagnostic test for vascular diseases is duplex ultrasonography (DUS) test. DUS testing is often chosen due to the widespread availability of ultrasound machines, negligible side effects of the procedure, low procedural costs, and therefore nearly unlimited repeatability on a given patient. DUS testing displays two-dimensional (2-D) vessel shapes in brightness mode (B-mode) and measures velocity waveforms in motion mode (M-mode). This modality and its nomenclature are known to those skilled in the art and therefore are not discussed in any detail here. However, as DUS testing provides only 2-D B-mode longitudinal images of arterial structures with relatively low resolution, it remains insufficient in its current state of application and methodology to provide the medical imagery required to conduct ICHD in which 3-D blood flow is simulated.

DUS testing could be used for applications in ICHD if two concerns were addressed. First, images produced from DUS B-mode testing must be capable of providing 3-D morphological geometries of diseased arterial and cardiovascular system. Second, if so, the 3-D geometries must be equivalent or effectively equivalent to those constructed from CTA scans. If both concerns could be satisfactorily addressed, DUS B-mode images could be used for applications in ICHD and therefore directly facilitate more widespread and timely diagnosis and treatment of vascular disease. Additionally, DUS B-mode images could ideally be used for preventative screening of potential abnormalities or for monitoring the progression of existing stenosis or aneurysm through annual physical exams, thus significantly promoting public health.

It would therefore be advantageous if a method were provided which produced 3-D morphological geometries from DUS B-mode images that were of equivalent quality to those derived from CTA scans, thereby facilitating more widespread application of ICHD and conferring diagnostic and treatment advantages in cardiovascular diseases without the risks and disadvantages associated with CTA scans.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, methods and systems capable of performing medical imaging and corresponding 3-D construction in support of cardiovascular diagnosis and treatment.

According to a nonlimiting aspect of the invention, a method is provided for acquiring duplex-ultrasound (DUS) cine loop images and producing three-dimensional arterial morphological geometries from duplex-ultrasound brightness-mode images extracted from the duplex-ultrasound cine loop images. The method includes acquiring two-dimensional brightness-mode images of arterial systems along both longitudinal and transverse directions of an artery via ultrasound cine looping, and constructing three-dimensional arterial morphological geometries from the two-dimensional brightness-mode images via an image-based deep-learning technique. The three-dimensional arterial morphological geometries are sufficiently detailed for use in image-based computational hemodynamics applications.

According to another nonlimiting aspect of the invention, a system is provided that acquiring two-dimensional brightness-mode images of arterial systems along both longitudinal and transverse directions of an artery via ultrasound cine looping, and constructing three-dimensional arterial morphological geometries from the two-dimensional brightness-mode images via an image-based deep-learning technique. The three-dimensional arterial morphological geometries are sufficiently detailed for use in image-based computational hemodynamics applications.

These and other aspects, arrangements, features, and/or technical effects will become apparent upon detailed inspection of the figures and the following description.

### BRIEF DESCRIPTION OF THE DRAWINGS

FIG. 1 is a graphical depiction of an example of an ICHD process to gain hemodynamic indicators for carotid arterial disease from medical images.

FIG. 2 is a schematic representation of calculating FFR from local pressure measurements taken on proximal and distal sides of a stenosis.

FIGS. 3A and 3B are 3-D constructions of carotid arteries from CTA (FIG. 3A) and MRA (FIG. 3B).

FIGS. 4A and 4B represent B-mode longitudinal images without (FIG. 4A) and with artery walls labeled (FIG. 4B), FIGS. 4C and 4D represent B-mode transverse images before bifurcation without (FIG. 4C) and with artery walls labeled (FIG. 4D), and FIGS. 4E and 4F represent B-mode transverse images after bifurcation without (FIG. 4E) and with artery walls labeled (FIG. 4F).

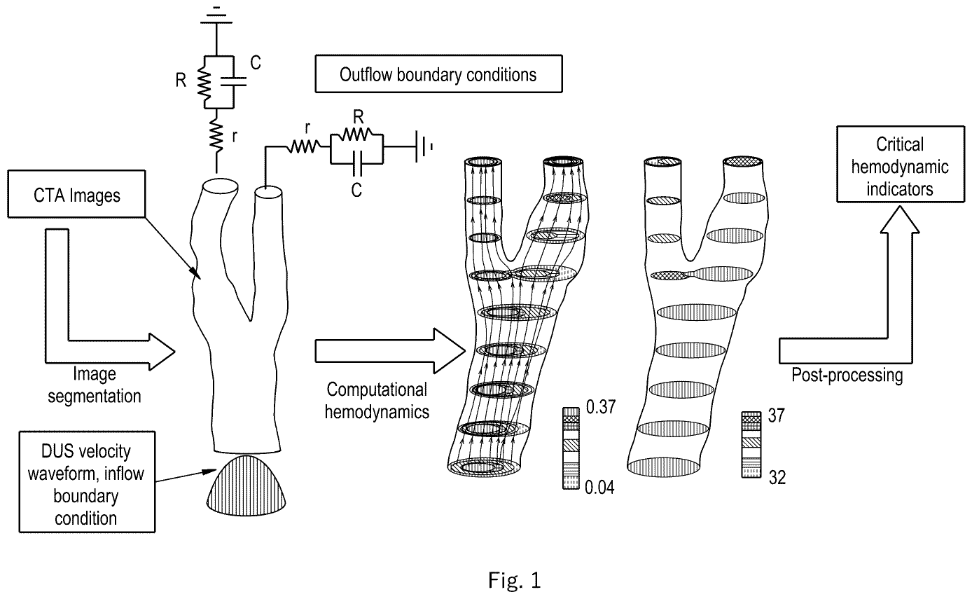

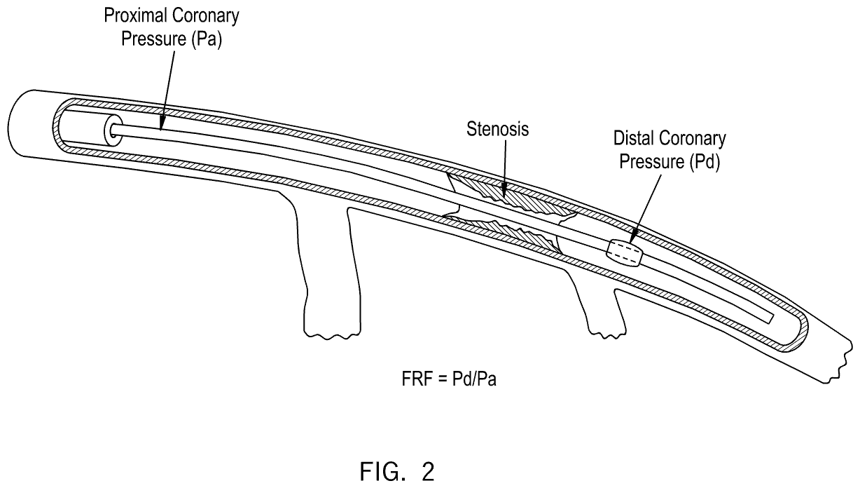

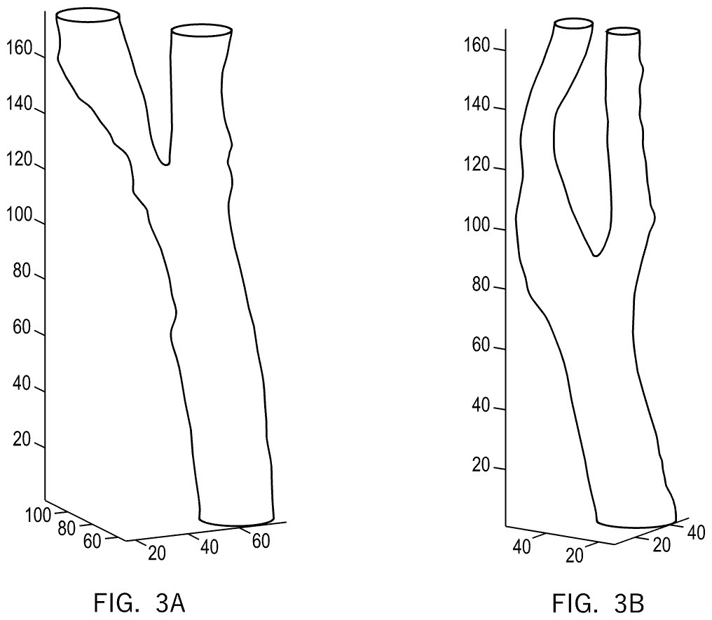

### DETAILED DESCRIPTION OF THE INVENTION

The intended purpose of the following detailed description of the invention and the phraseology and terminology employed therein is to describe what is shown in the drawings, which depict and/or relate to one or more nonlimiting embodiments of the invention, and to describe certain but not all aspects of the embodiment(s) depicted in the drawings. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended provisional claims, and not the detailed description, are intended to particularly point out subject matter regarded to be aspects of the invention, including certain but not necessarily all of the aspects and alternatives described in the detailed description.

The following describes methods of constructing 3-D arterial morphological geometries of cardiovascular systems from DUS B-mode images, wherein the constructed 3-D arterial morphological geometries are effective for use in ICHD methodology. Generally, such a method includes acquiring B-mode images in both longitudinal and transverse directions of an artery via DUS cine loops, and using an image-based deep-learning model utilizing computational programs to construct the 3-D arterial morphological geometries.

Cine looping is an existing function of medical ultrasound devices that is well known to those skilled in the art and therefore is not discussed in any detail here. In brief, cine loops are sequential sequences of ultrasound images which are used to capture motion. An ultrasound cine looping methodology known in the art includes the acquisition of ultrasound cine loops through a neck probing, in longitudinal and transverse directions of an artery, and video recording. By obtaining B-mode images in two directions, 3-D construction of images is enabled. Ultrasound cine looping allows video recording of B-mode images in longitudinal and transverse directions of an artery through a uniform movement of the probe, thereby enabling construction of 3-D images.

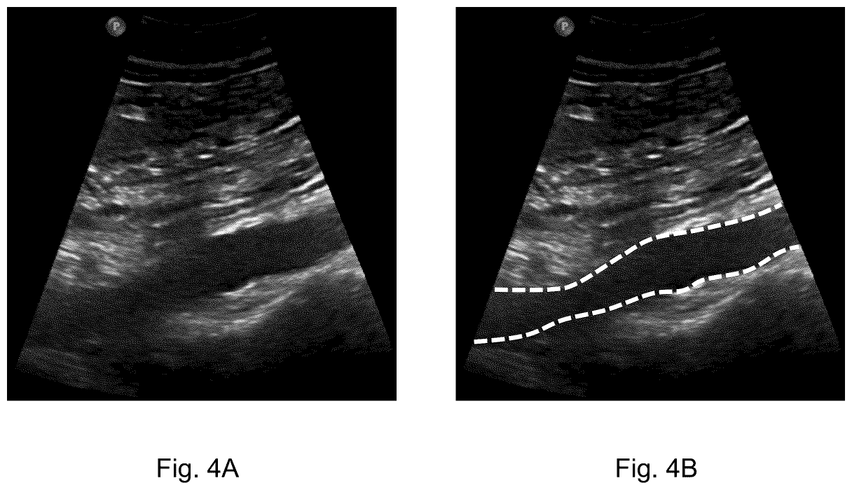

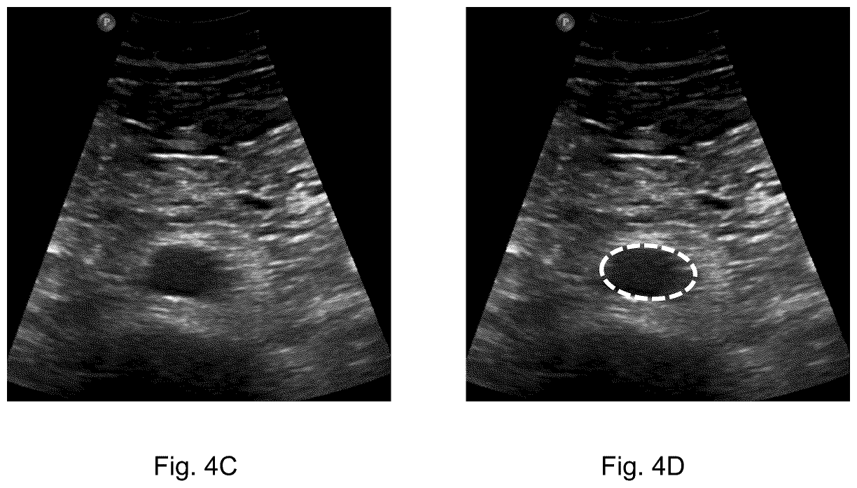

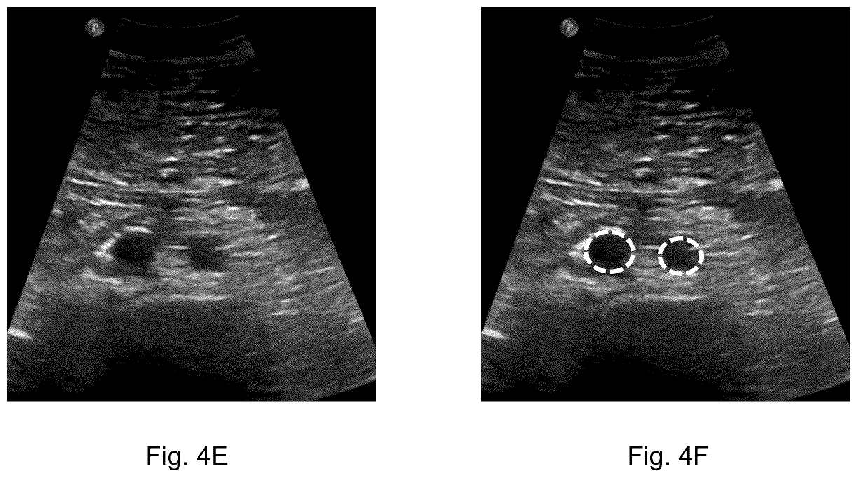

Relating to the present invention, cine loops can be used to produce images from which a user or a computational program may capture and analyze the motion of arterial walls. Such a computational program can extract B-mode images from the cine loops, then identify and label artery walls on the B-mode images. According to a nonlimiting aspect of the invention, a cine loop sweep can be carried out by ultrasound professionals along the neck in longitudinal or transverse directions of an artery and recorded as a video, as known by those skilled in the art. As an example, two or three longitudinal B-mode images and about 500 to 1000 transverse B-mode images can be captured on each of the left and right side of a patient. FIGS. 4A and 4B represent B-mode longitudinal images without artery walls labeled (FIG. 4A) and with artery walls labeled (FIG. 4B), FIGS. 4C and 4D represent B-mode transverse images before bifurcation without artery walls labeled (FIG. 4C) and with artery walls labeled (FIG. 4D), and FIGS. 4E and 4F represent B-mode transverse images after bifurcation without artery walls labeled (FIG. 4E) and with artery walls labeled (FIG. 4F).

A computational toolkit can be used to provide Graphic Processing Unit (GPU)-parallelized 3-D anatomical geometries extraction of arteries from both existing 3-D medical imagery, including CTA and MRA images, and imagery derived from DUS cine loops. The constructed 3-D geometry from such a toolkit has been established to be suitable for use in ICHD applications, as it has been proven effective using CTA-and MRA-based 3-D images to quantify trans-stenotic pressure gradients for arterial stenoses.

A deep-learning model capable of use with methods of this invention may be adapted from previous deep-learning algorithms for various medical image computing applications, including cell/nuclei segmentation and recognition, 3-D pancreas segmentation, 2-D/3-D liver lesion detection, 3-D pulmonary sarcoidosis localization, content-based image retrieval, disease classification and diagnosis, and other related tasks. Furthermore, the deep-learning model may be refined as experimental data is acquired. Deep learning methodologies and applications, as applied to computational programs, are well known in the art and therefore are not discussed in any detail here.

The deep-learning model may utilize a deep encoder-decoder neural network based on convolutional networks adapted for biomedical image segmentation, as known to those skilled in the art. Such convolutional networks demonstrate exceptional segmentation performance in various types of medical images. Furthermore, the neural network may incorporate two attention modules in a convolutional network architecture in order to ensure robust DUS image segmentation. Such a neural network takes as input the longitudinal and transverse B-mode images and conduct efficient model inference for image segmentation in an end-to-end, pixel-to-pixel manner.

A neural network as described above is preferably capable of providing certain capabilities. For example, it provides multi-view image inputs, specifically multiple transverse and longitudinal B-mode images, in order to provide contextual information. Such input provides contextual information, for example the spatial configuration of carotid arteries and other organs, for feature representation learning such that pixel prediction is not isolated from other regions. This is often critical to filter out pixel noise inherent in most DUS images.

Second, it includes a spatial attention gate on each skip connection to emphasize salient feature regions for artery localization; in the present embodiment, carotid artery localization. This gate suppresses feature activations in irrelevant image regions. The attention gate is a smaller, neural sub-network which calculates a scaling coefficient for each feature by analyzing both the activations and the context, and then applying the coefficient to multiply the corresponding feature in the input feature maps. Because fully convolutional operations preserve the spatial distribution of objects in input images, the coefficients can be encouraged to identify artery regions and prune image background and noise. The attention gate can be placed at each skip connection linking the different-scaled outputs of the encoder to the decoder, such that the attention mechanism is incorporated into feature learning at different levels and addresses scale variation in arteries while preserving the local details.

Finally, it includes a channel-wise attention block on top of the encoder to selectively highlight class-dependent feature maps for carotid artery segmentation. The channel-wise attention block operates using a self-attention module on channels of feature maps by using squeeze-excitation learning. First, the spatial information in a feature map can be “squeezed” into a channel descriptor before channel-wise statistics for the feature map are generated using global average pooling (GAP). Finally, an expectation operator may be used to map the globally aggregated information to a set of scale factors, which are used to emphasize the class-dependent feature maps for discriminability enhancement of certain features of an image. This GAP-based attention may be achieved by applying a two-layer, fully connected network to scale generation and then conducting a channel-wise multiplication between the scale factors and input feature maps. In this way, the network is capable of capturing feature statistics and model complex data structures.

The neural network is preferably capable of automatically learning image processing rules from exemplars, or training data. For each training image (longitudinal or transverse), its associated label may be a binary mask, with ones for arteries and zeroes for other regions. With these training images and corresponding labels, the neural network can be learned with standard backpropagation, a process known to those skilled in art, by minimizing a linear combination of a cross-entropy loss and a Dice loss. Because carotid arteries usually account for only a small proportion of each training image, the pixels in carotid arteries should be highlighted to avoid trivial solutions. Therefore, higher weights may be employed to emphasize those carotid artery regions in the cross-entropy loss and further combine it with a Dice loss to deal with the data imbalance. Extensive data augmentation may be applied to model training, including random image rotation, shearing, translation, elastic deformation, and intensity adjustment. For each new, unseen testing image, the neural network can preferably learn knowledge to predict one output map, where pixels corresponding to carotid arteries exhibit higher response values than the others. With image segmentation in longitudinal and transverse views, the deep-learning model is capable of constructing carotid arteries to approximate the 3-D geometry by using the corresponding positions of sampling points between the two view images.

3-D arterial morphological geometries constructed as described above may be validated by being compared against 3-D geometries constructed based on CTA scans. CTA scans remain the gold standard of medical imaging in arterial and cardiovascular applications. Disadvantages associated with CTA scanning are not a result of the quality of CTA-derived images but rather of the risks and costs associated with CTA scans and acquiring such images. In order to validate the 3-D arterial morphological geometries constructed by methods disclosed herein, an additional step of comparing DUS 2-D image-based geometries of the present method to CTA scan-based geometries can be performed.

The image data collected with a method as described above can be similar to those produced from CTA imaging, as the image data herein can be acquired from cine loops along three different directions. The deep learning methodology is preferably capable of overcoming the relatively low resolution of DUS images, and a construction toolkit presently used for CTA-based 3-D geometry construction can be adapted for use with DUS images. Nevertheless, shape comparison may be utilized in order to compare the 3-D geometries derived from either source.

The method of comparing 3-D geometries generally comprises constructing 3-D morphological geometries from both DUS-based images and CTA-based images using the aforementioned process, comparing the lumen area at representative locations from DUS-based and CTA-based images, and estimating the errors using uncertainty quantification analysis.

A system for accomplishing a method encompassing the aforementioned aspects of the invention may be integrated into ultrasound machines, 3-D printers, computational solvers, or other apparatuses which render the 3-D geometries produced interpretable by medical professionals, thereby facilitating diagnosis and treatment of cardiovascular diseases. Specifically, such a system may produce the 3-D geometries in Standard Tesselation Language (STL) files for use with computer-aided design (CAD) programs, 3-D printers to enable experimental measurement, or input into additional computational solvers.

According to another nonlimiting aspect of the invention, a system is provided that constructs 3-D arterial morphological geometries from 2-D DUS B-mode images by first acquiring B-mode images in both longitudinal and transverse directions of an artery via DUS cine loops, and then using an image-based deep-learning model to construct the 3-D arterial morphological geometries.

As previously noted above, though the foregoing detailed description describes certain aspects of one or more particular embodiments of the invention, alternatives could be adopted by one skilled in the art. It should be noted that, although much of the current terminology and experimental substantiation of the present invention focuses on carotid arterial stenosis, the method and application of the present invention may be applied to other vasculature and many different vascular diseases and arterial analyses. As such, and again as was previously noted, it should be understood that the invention is not necessarily limited to any particular embodiment described herein or illustrated in the drawings.

1. A method of acquiring duplex-ultrasound (DUS) cine loop images and producing three-dimensional arterial morphological geometries from duplex-ultrasound brightness-mode images extracted from the duplex-ultrasound cine loop images, the method comprising:
acquiring two-dimensional brightness-mode images of arterial systems along both longitudinal (coronal and sagittal) and transverse (axial) directions of an artery via ultrasound cine looping; and
constructing three-dimensional arterial morphological geometries from the two-dimensional brightness-mode images via an image-based deep-learning technique;
wherein the three-dimensional arterial morphological geometries are sufficiently detailed for use in image-based computational hemodynamics applications.

2. A method according to claim 1, wherein the three-dimensional arterial morphological geometries are produced by a computational toolkit.

3. A method according to claim 1, wherein the three-dimensional arterial morphological geometries are provided in a format that directly feeds into image-based computational hemodynamic applications.

4. A method according to claim 1, wherein the deep-learning model identifies arterial walls in brightness-mode images in order to construct the three-dimensional arterial morphological geometries.

5. A method according to claim 1, wherein the deep-learning model utilizes an encoder-decoder neural network.

6. A method according to claim 5, wherein the encoder-decoder neural network incorporates two attention modules into the encoder-decoder neural network architecture.

7. A method according to claim 5, wherein the encoder-decoder neural network takes as input images from multiple views.

8. A method according to claim 5, wherein the encoder-decoder neural network utilizes a spatial attention gate which emphasizes salient features.

9. A method according to claim 8, wherein the spatial attention gate calculates a scaling coefficient for each feature by analyzing activations and context and then applying the scaling coefficient to multiply the corresponding feature in input feature maps.

10. A method according to claim 5, wherein the encoder-decoder neural network utilizes a channel-wise attention module.

11. A method according to claim 10, wherein the channel-wise attention module operates based on a squeeze-excitation learning method, the method comprising:
compressing spatial information in feature maps into a channel description;
generating channel-wise statistics for feature-maps using global-average pooling; and
mapping globally aggregated information to a set of scale factors by exploiting an excitation operator, wherein the scale factors emphasize class-dependent feature maps for discriminability enhancement of certain features of an image.

12. A method according to claim 5, wherein the encoder-decoder neural network learns image processing rules from training data, wherein training images are labeled with a binary mask.

13. The method of claim 1, wherein the method provides the three-dimensional arterial morphological geometries in Standard Tesselation Language format.

14. The method of claim 1, further comprising using the method to identify and diagnose carotid artery stenosis.

15. The method of claim 1, wherein the three-dimensional arterial morphological geometries are used in image-based computational hemodynamics applications by:
constructing the three-dimensional arterial morphological geometries from the same patient and the same method but from both the duplex-ultrasound brightness-mode images and computed angiography-based images;
comparing the three-dimensional arterial morphological geometries from the duplex-ultrasound brightness-mode images and the computed angiography-based images; and
estimating errors using uncertainty quantification analysis.

16. A method of any of the preceding claims, wherein the method is performed by a system integrated into an ultrasound machine, a three-dimensional printer, and/or a computational solver.

17. A system for producing three-dimensional arterial morphological geometries from duplex-ultrasound brightness-mode images, the system comprising:
means for acquiring two-dimensional brightness-mode images of arterial systems along both longitudinal and transverse directions of an artery via ultrasound cine looping; and
means for constructing three-dimensional arterial morphological geometries from the two-dimensional brightness-mode images via an image-based deep-learning model;
wherein the three-dimensional arterial morphological geometries are sufficiently detailed for use in image-based computational hemodynamics applications.
